SEQUENCE LISTING (6 sequences)

SEQ ID NO: 1 - DNA, 2572 bp, Artificial Sequence
  synthesized in laboratory
tccctcctttaattattttttctattgtttctttattctttgttttgccatttataaacctgtagaaaagtttgggttcgtctttgcttttattcaccacacctttctcaaagtttctttattcctctctccttactcttaatatatattcatttcttgtatccctgtactgtcatctgttattatcatttctctgctttatcagtttcttccaagcttttgcacctttttagcttgtatgaatctagcattgtaccaagcatatatttttttcttaattctataaacaagtacaaattttttcatgcctacattatatttctgtaagaatatttcatatttcatattgtctttccatacgtaatatcaatatcagcaaaacagacccttaatcattcaaaatccactcatgcacaatttaatctctctcctttgtagtcatctctgtaacttatcccattctcctcttatatttgcatctctaatgtcatatgatcacttcttcccattggactaacatattgtatgattggaggtgactctggtttctttgtgaatactaggtcaagcaacgacagttccctcctgtaccttgttgacttctccacccattgattcattgcattaaccatggtcaattgtaacacttccttgatccactgcacagcattacccattactctcatctctctccattttacttttttgcagttaaaagtctccaactaaaagtattcttatgctttcatttacattcttacattaataccaagggcttgttagtggtgaaaatatctggttgtaaacaaagcgctggcctctccctctccactgccaccattgttctgttctgataccattttaccggtgaaagaaaattgctggtattccagtatactagataccagtgcacatacctagccctaccacagtcttaagaaaaacaacattcttctgcattctgttggtagtttttaattcaaaaacttataatggcaccaaagaggcttattggtggtgaaaataaggaatctcgtaaaagtgtgaaagtgcaagtgttggtgagccacagccctccactaatgagttcaaaggaaccacaccagacattttcatggatggtgactcgtcctctgatgacctccctcctcctcccctcttcttctacgtcatccatcaccagctttcacttatggtatgtacaaatcaaaatcataaaaaaaaatttacattgaaattttcataaacttgaggttttgctaagattagaagaaataacctgatttataagtatcaatagtcaaactttttgatacccaaacagtcatttggaattaattaagtttgagtattgagtatccactacatatttaggttttatctaacattgttgctgtgatactggtatgaaggcaacatggctagaaaggggtgtagcaaacaagttgtgaaggggaggtgtagtgtacaaaaatctaatatgaaatgagagataaaaatagataaaagttacaaacctaggtctacttttccactgttttcataaaaaacaatatcagatacacatttgttttacttcacatggtaaaacatcatttacaatacttctcattgatactgagggaatgaaatagagaagtaatactaatcaccaagataaggcagtcatgtctcaaaagatactccttctctgttgttgttcattatcatgacactttaaataatgtgtttgtttaattttagatggcaatatctttcattataatttgtaatgaattttttaggggatgaaaatagcatgcacaaaacaccctataatatgtacaaatcacccccactatgggagtaattcatacaaactggatttttttaatcaaatggcaaaaacaagatcactaattttgtcctgataaatttagctgtcataggtagtccataaattgaagattgcttctgatccatagtcttcaagatttaaatctgatttctcagtaatattttttcactaatagtataatatacctctatgtcaccctacctctcctcttttgacacattcaatattacattctggacaaacatataatccaccctcacttcactctttcactcctatcttaacctagtactcatctaaatcctcttgcttacatacagtattctacatcacaagctcttactgaaatcatacctgtatatttcactggaccaataacaattccattctttcaataatcatctgctttcatacaaattcagtgaaaaatattgcatacccattccatatacatcttaaaaggatagcattcatgtaatttccattgccttttttattacaaaataaaaaaaaaagaaattcacagcaagaactcacccttaatctatgatctgccactgtccagactctgctggcaaagtcattggaggcaccaagaatcaatgattcctcagcatcaaaatcaatagctgttactcctgcattgctgcctgtcaatatgcctctggcttccactgtggctgtaaggagtaataaaaaaatttagagagagagagagagagagagagagagaga

SEQ ID NO: 2 - DNA, 25 bp, Artificial Sequence
  synthesized in laboratory
ttaagtttgagtattgagtatccac

SEQ ID NO: 3 - DNA, 25 bp, Artificial Sequence
  synthesized in laboratory
aatgagaagtattgtaaatgatgtt

SEQ ID NO: 4 - DNA, 45 bp, Artificial Sequence
  synthesized in laboratory
gaaggtgaccaagttcatgctatttttgtacactacacctccccc

SEQ ID NO: 5 - DNA, 42 bp, Artificial Sequence
  synthesized in laboratory
gaaggtcggagtcaacggatttttgtacactacacctcccct

SEQ ID NO: 6 - DNA, 27 bp, Artificial Sequence
  synthesized in laboratory
gctagaaagggrtgtagcaaacaagtt

(19) United States
(12) Patent Application Publication
(10) Pub. No.: US 2021/0332433 A1
(43) Pub. Date: Oct. 28, 2021

(54) Method for identifying genetic sex of Portunus trituberculatus

(71) Applicant: Ningbo University, Ningbo (CN)

(72) Inventors: Ronghua Li, Ningbo (CN); Junkai Lu, Ningbo (CN); Zhouyi Zhang, Ningbo (CN); Weijia Zhang, Ningbo (CN); Chunlin Wang, Ningbo (CN); Changkao Mu, Ningbo (CN); Weiwei Song, Ningbo (CN)

(73) Assignee: Ningbo University

(21) Appl. No.: 17/194,252

(22) Filed: Mar. 6, 2021

(30) Foreign Application Priority Data
Mar. 6, 2020 (CN) 202010152999.7
Mar. 13, 2020 (CN) 202010177021.6
Mar. 13, 2020 (CN) 202010177023.5

(51) Int. Cl. C12Q 1/6879; C12Q 1/6858

(52) U.S. Cl. CPC C12Q 1/6879; C12Q 2600/156; C12Q 1/6858

(57) ABSTRACT

A method for identifying the genetic sex of Portunus trituberculatus is provided. The SNP markers provided by the present invention presents biallelic heterozygosity in male individuals, but presents homozygous allele type in female individuals, and can be detected by PCR amplification and sequencing method, HRM method or KASP method. The detection method is flexible and the results are stable. Among them, the KASP and HRM detection methods have high throughput, fast detection speed, and simple application. It provides an important tool for the breeding of all-female population of P. trituberculatus.

### CROSS REFERENCE OF RELATED APPLICATION

The present application claims priority under 35 U.S.C. 119(a-d) to CN 202010177023.5, filed Mar. 13, 2020; CN 202010152999.7, filed Mar. 6, 2020; and CN 202010177021.6, filed Mar. 13, 2020.

### BACKGROUND OF THE PRESENT INVENTION

### Field of Invention

The invention belongs to the technical field of genetic breeding of aquaculture animals, and specifically relates to a method for identifying the genetic sex of Portunus trituberculatus.

### Description of Related Arts

Portunus trituberculatus, commonly known as swimming crab or white crab, belongs to the Crustacea, Decapoda, Portunus, which is widely distributed in the southeastern coast of China and waters such as Japan, Korea, and the Malaysian archipelago, is an important economic crustacean for fishing and breeding. According to data from the “China Fishery Statistical Yearbook of 2019”, in 2018, the farming production of P. trituberculatus reached 116,251 tons in China. It is an economically important large-scale marine crab. China has started the artificial breeding of P. trituberculatus since 1990s. Because of its good taste and rich nutrition, it has been loved by the consumers, and female crabs that reached sexual maturation are more profitable with a higher market value due to the accumulation of vitellogenin in the ovary. Therefore, the production of monosex females of P. trituberculatus has a good market prospect. The development of sex-linked molecular markers is conducive to the rapid identification of sex-reversed individuals and speeds up the breeding process of all-female P. trituberculatus.

Molecular marker technology is an effective tool to identify genetic sex. Single nucleotide polymorphism (SNP) as a third-generation molecular marker has been widely used in genotyping. Competitive allele-specific PCR (kompetitive allele-specific PCR, KASP) is one of single SNP genotyping methods based on allele-specific oligonucleotide extension and fluorescence resonance energy transfer (FRET) signals has become a global benchmark technology with the characteristics of high throughput, low cost, high sensitivity and specificity.

### SUMMARY OF THE PRESENT INVENTION

The present invention provides a method for identifying the genetic sex of P. trituberculatus, in which molecular markers for identifying the sex of P. trituberculatus are used, which can quickly perform high-throughput gender identification, thereby making up for the deficiencies of the existing technology.

The present invention first provides a first SNP site PtS1 as a genetic sex identification marker for P. trituberculatus; wherein the SNP site is a fragment of SEQ ID NO:1; or SEQ ID NO:1 by substituting, deleting, or adding one or more nucleotides; wherein the at position 1452 of the complementary fragment derived from SEQ ID NO:1, is 1452A>G.

Another aspect of the present invention provides a method for detecting the male and female genetic sex of P. trituberculatus, which is to distinguish the male and female sex of P. trituberculatus by detecting the above-mentioned SNP sites.

One of the specific methods is to perform detection by PCR amplification and sequencing methods. The sequencing results of the tested individuals are compared with the molecularly labeled DNA sequence. If the sequencing peak pattern of the tested individual is consistent with the molecularly labeled DNA sequence, and the SNP position is a single peak, the tested individual is female; if the sequencing peak diagram exhibits double peaks at the SNP position, it is male.

Another specific method is to detect by the KASP method, and determine the genotype of the sex-linked locus of P. trituberculatus based on the fluorescent signal to identify the genetic sex of the sample.

The present invention also provides a PCR detection product for sex identification of P. trituberculatus, and the PCR detection product is used to detect the above-mentioned SNP site.

One of the molecular reagents is a reagent for PCR amplification and sequencing method; the other is a reagent for KASP detection method;

wherein the reagents used for the PCR amplification and sequencing method include a primer pair for PCR amplification and sequencing for detecting the above SNP site PtS1, and the sequence information is as follows:

PS7 Forward:
(SEQ ID NO: 2
5′ -TTAAGTTTGAGTATTGAGTATCCAC- 3′;
PS7 Reverse:
(SEQ ID NO: 3)
5′ -AATGAGAAGTATTGTAAATGATGTT- 3′;

The reagents used in the KASP detection method include the following primers:

PtS1FAM (SEQ ID NO: 4):
GAAGGTGACCAAGTTCATGCTATTTTTGTACACTACACCTCCCCC,
PtS1HEX (SEQ ID NO: 5):
GAAGGTCGGAGTCAACGGATTTTTGTACACTACACCTCCCCT,
PtS1C (SEQ ID NO: 6):
GCTAGAAAGGGRTGTAGCAAACAAGTT;

The present invention also provides a second sex identification marker for P. trituberculatus, which is distributed in the sequence SEQ ID NO:7

The 6 sex-linked SNP sites on the ID NO:7 fragment are 153C>T, 185G>A, 214T>C, 236T>(A,C), 251T>C, 261A>T;

The present invention also provides a molecular reagent for genetic sex identification of P. trituberculatus, and the reagent is used to detect the aforementioned sex identification markers of P. trituberculatus;

One of the molecular reagents is a reagent for PCR amplification and sequencing method; the other is a reagent for high-resolution melting curve detection method;

The present invention also provides a primer pair (PS_11) for detecting the above-mentioned marker, the sequence of the upstream primer is:

(SEQ ID NO: 8)
5′-CCGACAACACAGATCCACTAAC-3′;

The sequence of the downstream primer is:

(SEQ ID NO: 9)
5′-CGAGTGTGGAGAGAATGATTTTT-3′;

Another aspect of the present invention provides a method for detecting the male and female genetic sex of P. trituberculatus, which is to distinguish the male and female sex of P. trituberculatus by detecting the sexidentification markers of P. trituberculatus;

One of the specific methods is to perform detection by PCR amplification and sequencing methods. The sequencing results of the tested individuals are compared with the molecularly labeled DNA sequence. If the sequencing peak pattern of the tested individual is consistent with the molecularly labeled DNA sequence, and each of the SNP positions exhibit a single peak, the tested individual is female, and if the sequencing peak diagram exhibits double peaks in all the SNP positions, it is male;

Another specific method is to detect by a high-resolution melting curve method, where the peak value of the male melting curve is at 80.52° C. and the peak value of the female melting curve is at 81.91° C.

The present invention also provides a third SNP site PtS2 as a genetic sex identification marker for P. trituberculatus, comprising a sex-linked SNP site at position 878 of SEQ ID NO: 10, and with a base T>C;

The present invention also provides a molecular reagent for genetic sexidentification of P. trituberculatus, and the reagent is used for detecting the above-mentioned SNP site;

One of the molecular reagents is a reagent for PCR amplification and sequencing method; the other is a reagent for KASP detection method;

The reagents used in the PCR amplification and sequencing method include primer pairs for PCR amplification and sequencing used to detect the above-mentioned SNP sites, and the sequence information is as follows:

PS8 Forward:
(SEQ ID NO: 11
5′ -ATACCAGACAAGAGGGCTTC- 3′;
PS8 Reverse
(SEQ ID NO: 12
5′ -TCCCATATAGATATTAGTGTCATTC- 3′;

The reagents used in the KASP detection method include the following primers:

PtS2FAM (SEQ ID NO: 13):
GAAGGTGACCAAGTTCATGCTAGGCTAGTGCACTGATCCTCCA;
PtS2HEX (SEQ ID NO: 14):
GAAGGTCGGAGTCAACGGATTGGCTAGTGCACTGATCCTCCG;
PtS2C (SEQ ID NO: 15):
CTGTCAACTTCACCTCAAGTTGATGTTA;

Another aspect of the present invention provides a method for detecting the male and female genetic sex of P. trituberculatus, which is to distinguish the male and female sex of P. trituberculatus by detecting the aforementioned SNP sites;

One of the specific methods is to perform detection by PCR amplification and sequencing methods. The sequencing results of the tested individuals are compared with the molecularly labeled DNA sequence. If the sequencing peak pattern is consistent with the molecularly labeled DNA sequence, and the SNP position exhibits a single peak, the tested individual is female; if the sequencing peak diagram exhibits double peaks in the

SNP position, it is male;

Another specific method is to use the KASP method to detect the genotype of the sex-linked locus of P. trituberculatus based on the fluorescent signal to identify the genetic sex of the sample.

The SNP markers provided by the present invention presents biallelic heterozygosity in male individuals, but presents homozygous allele type in female individuals, and can be detected by PCR amplification and sequencing method, HRM method or KASP method. The detection method is flexible and the results are stable. Among them, the KASP and HRM detection methods have high throughput, fast speed, and simple application. It provides an important tool for the breeding of all-female population of P. trituberculatus.

### BRIEF DESCRIPTION OF THE DRAWINGS

FIG. 1 is the sequencing chromatograms of PS7 primers, the SNP site was indicated by arrow.

FIG. 2 is the KASP detection result of PtS1 sex marker; the NTC black dot is the negative control (H₂O).

FIG. 3: is the sequencing chromatograms of PS_11 primers, (A): female, (B): male.

FIG. 4 is the high-resolution melting curve of PS_11 primers, (a): female, (b): male.

FIG. 5 is the sequencing chromatograms of PS8 primers, the SNP site was indicated by arrow.

FIG. 6 is the KASP detection result of PtS2 sex marker, in which NTC black dots is the negative control (H₂O), and pink dots indicate unsuccessful amplification.

### DETAILED DESCRIPTION OF THE PREFERRED EMBODIMENT

The terms involved in the present invention are explained as follows:

SNP (single nucleotide polymorphism): single nucleotide polymorphism, or single base polymorphism;

HRM (high-resolution melting): High-resolution melting curve is a new gene analysis technology based on the melting temperature of single nucleotides to form different melting curves. It has extremely high sensitivity and can detect single bases. It has the characteristics of low cost, high throughput, fast speed, and high accuracy of results. It is one of the common methods for single nucleotide polymorphism analysis.

KASP (kompetitive allele-specific PCR): Competitive allele-specific PCR is a single SNP genotyping based on allele-specific oligonucleotide extension and fluorescence resonance energy transfer (FRET) signals method.

The present invention will be described in detail below in conjunction with embodiments and drawings.

### EXAMPLE 1

### Screening of PtS1 Marker and Primer Design

The applicant extracted DNA from 15 males and 15 females of P. trituberculatus, performed 2b-RAD simplified genome sequencing, combined with the gender records of the samples, and adopted relevant analysis methods to obtain gender-related SNP markers and nucleic acid fragments. Then, using the tag sequence to blast againstthe simplified genome library of P. trituberculatus which was established in the laboratory of the inventor, as a result, the long scaffold sequence scaffo1d1070277 containing the gender tag was finally screened, and the corresponding nucleotide sequence was SEQ ID NO:1, containing the gender-related SNP marker (PtS1). The SNP site is located on the complementary fragment of the fragment SEQ ID NO:1, position 1452, which is 1452A>G.

(SEQ ID NO: 1)
TCCCTCCTTTAATTATTTTTTCTATTGTTTCTTTATTCTTTGTTTTGCCAT
TTATAAACCTGTAGAAAAGTTTGGGTTCGTCTTTGCTTTTATTCACCACAC
CTTTCTCAAAGTTTCTTTATTCCTCTCTCCTTACTCTTAATATATATTCAT
TTCTTGTATCCCTGTACTGTCATCTGTTATTATCATTTCTCTGCTTTATCA
GTTTCTTCCAAGCTTTTGCACCTTTTTAGCTTGTATGAATCTAGCATTGTA
CCAAGCATATATTTTTTTCTTAATTCTATAAACAAGTACAAATTTTTTCAT
GCCTACATTATATTTCTGTAAGAATATTTCATATTTCATATTGTCTTTCCA
TACGTAATATCAATATCAGCAAAACAGACCCTTAATCATTCAAAATCCACT
CATGCACAATTTAATCTCTCTCCTTTGTAGTCATCTCTGTAACTTATCCCA
TTCTCCTCTTATATTTGCATCTCTAATGTCATATGATCACTTCTTCCCATT
GGACTAACATATTGTATGATTGGAGGTGACTCTGGTTTCTTTGTGAATACT
AGGTCAAGCAACGACAGTTCCCTCCTGTACCTTGTTGACTTCTCCACCCAT
TGATTCATTGCATTAACCATGGTCAATTGTAACACTTCCTTGATCCACTGC
ACAGCATTACCCATTACTCTCATCTCTCTCCATTTTACTTTTTTGCAGTTA
AAAGTCTCCAACTAAAAGTATTCTTATGCTTTCATTTACATTCTTACATTA
ATACCAAGGGCTTGTTAGTGGTGAAAATATCTGGTTGTAAACAAAGCGCTG
GCCTCTCCCTCTCCACTGCCACCATTGTTCTGTTCTGATACCATTTTACCG
GTGAAAGAAAATTGCTGGTATTCCAGTATACTAGATACCAGTGCACATACC
TAGCCCTACCACAGTCTTAAGAAAAACAACATTCTTCTGCATTCTGTTGGT
AGTTTTTAATTCAAAAACTTATAATGGCACCAAAGAGGCTTATTGGTGGTG
AAAATAAGGAATCTCGTAAAAGTGTGAAAGTGCAAGTGTTGGTGAGCCACA
GCCCTCCACTAATGAGTTCAAAGGAACCACACCAGACATTTTCATGGATGG
TGACTCGTCCTCTGATGACCTCCCTCCTCCTCCCCTCTTCTTCTACGTCAT
CCATCACCAGCTTTCACTTATGGTATGTACAAATCAAAATCATAAAAAAAA
ATTTACATTGAAATTTTCATAAACTTGAGGTTTTGCTAAGATTAGAAGAAA
TAACCTGATTTATAAGTATCAATAGTCAAACTTTTTGATACCCAAACAGTC
ATTTGGAATTAATTAAGTTTGAGTATTGAGTATCCACTACATATTTAGGTT
TTATCTAACATTGTTGCTGTGATACTGGTATGAAGGCAACATGGCTAGAAA
GGGGTGTAGCAAACAAGTTGTGAAGGGGAGGTGTAGTGTACAAAAATCTAA
TATGAAATGAGAGATAAAAATAGATAAAAGTTACAAACCTAGGTCTACTTT
TCCACTGTTTTCATAAAAAACAATATCAGATACACATTTGTTTTACTTCAC
ATGGTAAAACATCATTTACAATACTTCTCATTGATACTGAGGGAATGAAAT
AGAGAAGTAATACTAATCACCAAGATAAGGCAGTCATGTCTCAAAAGATAC
TCCTTCTCTGTTGTTGTTCATTATCATGACACTTTAAATAATGTGTTTGTT
TAATTTTAGATGGCAATATCTTTCATTATAATTTGTAATGAATTTTTTAGG
GGATGAAAATAGCATGCACAAAACACCCTATAATATGTACAAATCACCCCC
ACTATGGGAGTAATTCATACAAACTGGATTTTTTTAATCAAATGGCAAAAA
CAAGATCACTAATTTTGTCCTGATAAATTTAGCTGTCATAGGTAGTCCATA
AATTGAAGATTGCTTCTGATCCATAGTCTTCAAGATTTAAATCTGATTTCT
CAGTAATATTTTTTCACTAATAGTATAATATACCTCTATGTCACCCTACCT
CTCCTCTTTTGACACATTCAATATTACATTCTGGACAAACATATAATCCAC
CCTCACTTCACTCTTTCACTCCTATCTTAACCTAGTACTCATCTAAATCCT
CTTGCTTACATACAGTATTCTACATCACAAGCTCTTACTGAAATCATACCT
GTATATTTCACTGGACCAATAACAATTCCATTCTTTCAATAATCATCTGCT
TTCATACAAATTCAGTGAAAAATATTGCATACCCATTCCATATACATCTTA
AAAGGATAGCATTCATGTAATTTCCATTGCCTTTTTTATTACAAAATAAAA
AAAAAAGAAATTCACAGCAAGAACTCACCCTTAATCTATGATCTGCCACTG
TCCAGACTCTGCTGGCAAAGTCATTGGAGGCACCAAGAATCAATGATTCCT
CAGCATCAAAATCAATAGCTGTTACTCCTGCATTGCTGCCTGTCAATATGC
CTCTGGCTTCCACTGTGGCTGTAAGGAGTAATAAAAAAATTTAGAGAGAGA
GAGAGAGAGAGAGAGAGAGAGA;

Primer Premier 5 software was used to design primer PS7 for sex marker verification according to the sequence of scaffo1d1070277, the primer design should meet the following conditions: the target amplicon is between 100-300bp; the annealing temperature should be between 50-60° C.; mismatches, hairpin structure and primer dimer should be avoided between the forward and reverse primers.The primers were synthesized by Sangon Biotech (Shanghai) Co., Ltd. (Table 1).

TABLE 1
Information of sequencing primers
(annealing temperature Tₐ: 60° C.)

| Primer | | |
|---|---|---|
| ID | | Primer sequence |
| PS7 | Forward | 5′ -TTAAGTTTGAGTATTGAGTATCCAC- 3′ |
| | | (SEQ ID NO: 2) |
| | Reverse | 5′ -AATGAGAAGTATTGTAAATGATGTT- 3′ |
| | | (SEQ ID NO: 3) |

The KASP detection primers were designed according to the sequence information of the SNP site. Two allele-specific reverse primers (the 5′ends were respectively connected to specific sequences that can bind to the FAM fluorophore and HEX fluorophore) and a universal forward primer were designed respectively. The primer combinations for site PtS1 (located in scaffo1d1070277 sequence) are: PtS1FAM, PtS1HEX, PtS1C, and the primer sequences are shown in Table 2.

TABLE 2
KASP primer sequence information TABLE

| LocusID | Primer | Sequence (5′-3′) |
|---|---|---|
| PtS1 | PtS1FAM | GAAGGTGACCAAGTTCATGCTATTTTTG |
| | | TACACTACACCTCCCCC (SEQ ID NO: 4) |
| | PtS1HEX | GAAGGTCGGAGTCAACGGATTTTTGTA |
| | | CACTACACCTCCCCT (SEQ ID NO: 5) |
| | PtS1C | GCTAGAAAGGGRTGTAGCAAACAAGTT |
| | | (SEQ ID NO: 6) |

Note:
FAM specific sequence: GAAGGTGACCAAGTTCATGCT;
HEX specific sequence: GAAGGTCGGAGTCAACGGATT.

Sequencing method for genotyping detection

A total of 60 individuals (30 females +30 males) from a wild population of P. trituberculatus were collected, and the DNA of muscle was extracted using the tissue genomic DNA extraction kit (Bioteke, Beijing).

PS7 primers were used for PCR amplification and sequencing. At the same time, paraffin section and HE staining were performed on the sample gonad tissue to ensure the accuracy of gender identification.

The PCR reaction system is 100 μL, including: 100 ng DNA, 2×Power Taq PCR Master Mix (Bioteke, Beijing) 5 μL, 1 μmol of each primer.

The PCR amplification program is: denaturation at 94° C. for 3 min; 35 cycles of 94° C. for 1 min, 60° C. for 1 min, and 72° C. for 1 min. Finally, extend for 5min at 72° C. and store at 4° C. The PCR products were sent to Sangon Biotech (Shanghai) Co., Ltd. for sequencing in both directions. The sequencing results showed that there was a SNP site difference between male and female. Females are homozygous and males are heterozygous. The specific genotypes are presented in Table 3 and the sequencing diagram is presented in the FIG. 1.

TABLE 3
Genotype sequencing results of male and female
individuals of P. trituberculatus

| Primer | Genotype | Male | Female | Specificity (%) |
|---|---|---|---|---|
| PS7 | A/G | 27 | 1 | 93.3 |
| | A/A | 3 | 29 | |

Use KASP method for genotyping detection

In addition, 92 individuals (46 females +46 males) from the wild population of P. trituberculatus were collected, and the genomic DNA of the muscle tissue was extracted according to the instruction of the EZNA® tissue DNA kit.

The PtS1 locus was amplified and sequenced by PCR using the KASP primer combination, and the gonad tissue of the sample was paraffin sectioned and HE stained to ensure the accuracy of gender identification.

KASP reaction was carried out on Hydrocycler-16 (LGC Genomics, UK), the reaction system was including 0.5 μl 2×KASP 1536 Master Mix, 0.014 μl Primer mix (Primer mix includes 46 μl ddH₂O, 30 μl common primer (100 μM), 12 μl each of allele-specific primers (10004), 10 ng DNA. The Touch down PCR program is as follows: 94° C. for 10min; 10 touch-down cycles at 94° C. for 20s, 61-55 C for 10 s (−0.6° C. per cycle); 26 cycles at 94° C. for 20 s, 55° C. for 60s. Pherastar scanner (LGC Genomics, UK) was used for fluorescence detection of the reaction, and Kraken software (LGC Genomics, UK) was used for data analysis. If no genotyping is observed after the initial amplification, another 5-10 cycles could be added and analyzed again.

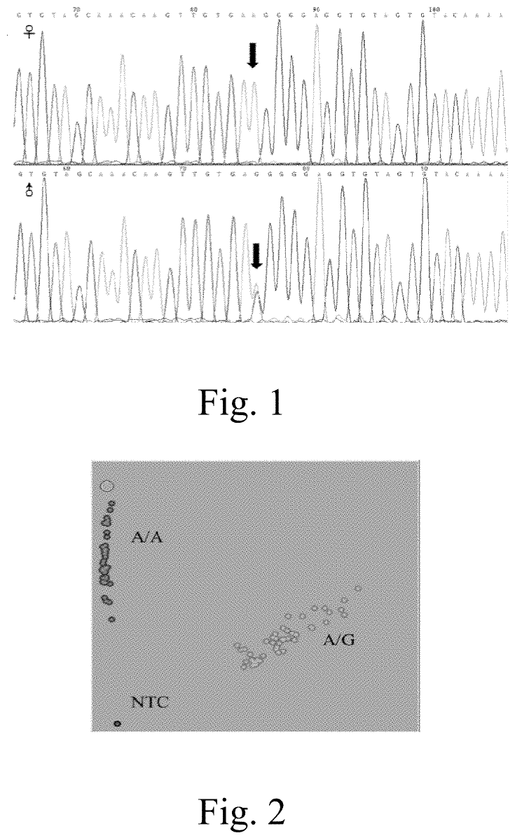

The genotyping results of 92 wild P. trituberculatus using KASP method are shown in FIG. 2. The typing results of PtS1 locus are homozygous A/A (red dot) and heterozygous A/G (green dot). The genotyping results of PtS1 are consistent with the phenotypic sex of each individual, and conform to the law of homozygous female and heterozygous male. PtS1 was successfully amplified in 92 individuals, and the accuracy of successfully amplified individuals was 100%.

### EXAMPLE 2

### Screening of Sex Marker and Primer Design of P. trituberculatus

DNA was extracted from 15 male and 15 female individuals of P. trituberculatus, and 2b-RAD simplified genome sequencing was performed, combined with the gender record of the sample, to obtain gender-related SNP markers and nucleic acid fragments. Then the tag sequence that contain the gender-related SNP was blasted against the simplified genome library of P. trituberculatus which was established in the laboratory of the inventor to obtain the long scaffold sequence (scaffo1d136081) containing the gender tag. Its nucleotide sequence is as follows (SEQ ID NO: 7):

AAATCACATTTAGAATACCATAACCTTTTACCTACTTCACAGCATGGCTTC
CGACAACACAGATCCACTAACACAGCTATAGCAACTTTAACAGAAGCAGTA
GCAAACTCTCTCACACAAAATAAGATCTGCACAATTGTTACAAGAGACATT
TCAAAAGCATTTGATAAAGTCTGGCATAACGAATTAAAATACAAACTAACA
AAACAACATCTCCACCCTATATACATCAAAATATTAAGCTCATTCCCAGAC
AACAGTTCAGGAAAAATCATTCTCTCCACACTCGAAGGGCAACCATTCCCA
CTATTAAGCGGCCTACCACAAGGAAGCAACTTATCCCCAACACTTTTCAAC
ATA;

Primer Premier 5 software was used to design the primers PS_11 according to the sequence of scaffo1d136081 for sex marker verification. The primer design should meet the following conditions: the target amplicon is between 100-300bp; the annealing temperature (Tₘ) should be between 50-60° C.; mismatches, hairpin structure and primer dimer should be avoided between the forward and reverse primers. The primers were synthesized by Sangon Biotech (Shanghai) Co., Ltd. see Table 4.

TABLE 4
Primer sequence information

| | | Annealing |
|---|---|---|
| Primer | | temperatue |
| name | Primer sequence (5′-3′) | (° C.) |
| PS_11 | 5′-CCGACAACACAGATCCACTAAC-3′ | 60 |
| | (SEQ ID NO: 8) | |
| | 5′-CGAGTGTGGAGAGAATGATTTTT-3′ | |
| | (SEQ ID NO: 9) | |

A total of 60 individuals (30 females+30 males) from a wild population of P. trituberculatus were collected, and the DNA of muscle was extracted using the tissue genomic DNA extraction kit (Bioteke, Beijing).

1. Use PS_11 Primers for PCR Amplification and Sequencing, and at the Same Time Perform Paraffin Section and HE Staining of the Sample Gonad Tissue to Ensure the Accuracy of Gender Identification.

The PCR reaction system is 10 μL, including: 100 ng DNA, 2×Power Taq PCR Master Mix (Bioteke, Beijing) 5 μL, 1 μmol of each primer.

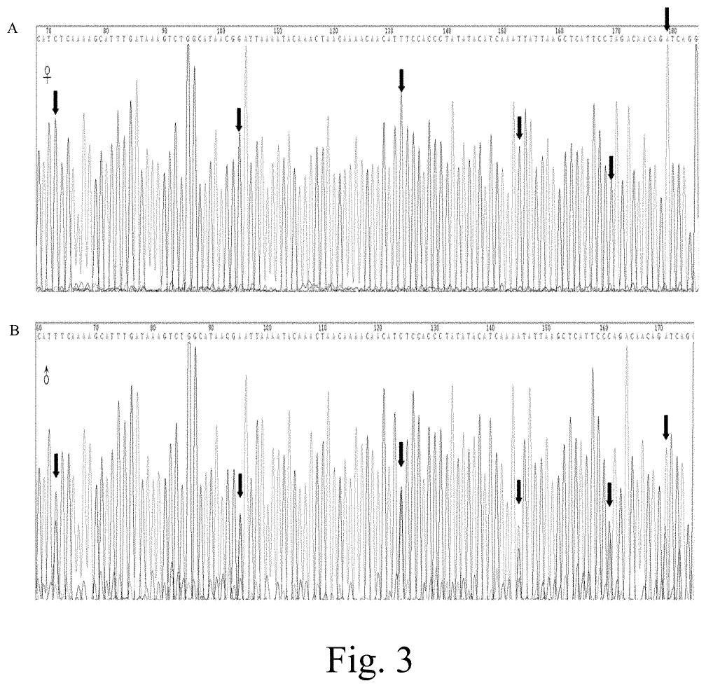

The PCR amplification program is: denaturation at 94° C. for 3 min; 35 cycles of 94° C. for 1 min, 60° C. for 1 min, 72° C. for 1 min. Finally, extend for 5min at 72° C. and store at 4° C. The PCR products were sent to Sangon Biotech (Shanghai) Co., Ltd. for sequencing in both directions . The sequencing results showed that there were differences in six SNP sites between males and females. Females were homozygous and males were heterozygous. The specific genotypes are shown in Table 5, and the sequencing diagram is shown in FIG. 3.

TABLE 5
Genotypes of male and female individuals of
P. trituberculatus

| Primer | | | | Specificity |
|---|---|---|---|---|
| name | Genotype | Male | Female | (%) |
| PS_11 | T/C; A/G; C/T; A/C; | 30 | 0 | 100.0 |
| | C/T; T/A | | | |
| | C/C; G/G; T/T; T/T; | 0 | 30 | |
| | T/T; A/A | | | |

2. Using HRM Curve for Genotyping Detection

The high-resolution melting curve method was tested for sex identification of P. trituberculatus using PS_11 primers . LightCycler®480 Saturated Fluorescent Dye HRM Kit was selected for the PCR amplification, which contains: 10 μl , 1×LightCycler® 480 HRM Master Mix with ResoLight® dye (Roche Diagnostics), 10 μmol of each primer, 30 ng DNA template, 1.6 μl, Mgcl₂, and make up to 20μL with water. The melting curve analysis and the PCR reaction were simultaneously performed on the LightCycler®480 real-time quantitative analyzer (Roche Diagnostics). The reaction procedure is as follows:

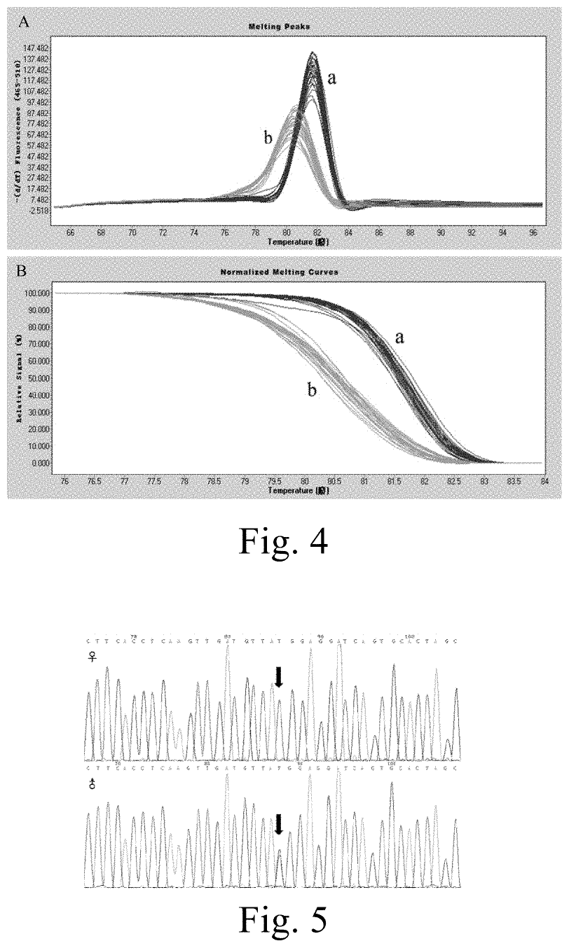

95° C. pre-denaturation for 5 minutes; 45 cycles of 95° C. denaturation for 30 s, annealing temperature for 30 s, 72° C. for 30 s. Data were analysed using the LightCycler®480 Gene Scanning Software v. 1.5. The results showed that the melting peak value of the male is 80.52° C., and the melting peak value of the female is 81.91° C. Females and males can be clearly distinguished in FIG. 4 This result proved that PS_11 primers can be used to effectively identify the genetic sex of P. trituberculatus.

### EXAMPLE 3

### Screening of PtS2 Marker and Primer Design

For 15 male and 15 female individuals of P. trituberculatus, DNA was extracted, 2b-RAD simplified genome sequencing was performed, combined with the gender record of the sample, and the correlation analysis method was used to obtain gender-related SNP markers and nucleic acid fragments. Then, using the tag sequence to blast against the simplified genome library of P. trituberculatus which was established in the laboratory of the inventor, as a result, the long fragment scaffold sequence scaffold325741 containing gender tags was finally screened, and the corresponding nucleotide sequence was SEQ ID NO:10, containing a gender-related SNP marker (PtS2). The sex-linked SNP is located at position 878 of SEQ ID NO: 10, which is T>C;

(SEQ ID NO: 10)
AAGGTTCCTTTCAAACACTGAGCCCTAGAACACTTCATATGACCCTCAGG
AAGTATTTTGTAATGTGTGCAGTAAGGTTCCTTTCAAACACTGAGCCCTAGAA
CACTTCATATGTCCCTCAGGAAGTATTTTGTAAGTAAATATAATAGAGTATAA
CATAAGTTTCAGTGGATAGTGCTAAAGGTGAAAGTACACGGATTTCTTGATTT
GCATATATTTGAAATATGCGATTTTGAAATAACACTATGTAAGAAAGAATCC
TTTTTCCATATTATGCACATTATATAGAATATGATGTCAAGAAAATCAAGTTG
CCCATCTGGAAACACCAGGTGACACCTGGTGTTCCCCCATGTGTACTGCCAAC
ATGCTGTCCCTTTGCACAGTGCTTCAAAGTAATTATCAGCAACAAAATCAGTG
TGCAAGTAACAATTTATCCATGCACCACTAGTTTGTTTGATATCCTTTAACTT
AATGTCAGCTGTGTTATTTGACTCACATACTTAACACAGACCAGCAATATCAC
ATCTATACAATGCATTGATAATAGAAAGGTACACATAGTAAATCAGATAGTT
TACCTTACCTTGGTACAAAAGCCAGCCCAGTACACTCGTCACTACCACCGACTT
ATCTAAGCCAACCATCCACAGCTGAGGACTAACTGATCCCACAACAAAACACA
TCACTGGTGATCCAAAGAATTAGACACATCATACAAATACATCACCCTCTCCA
CCTCAACAATTTACCACCATACCAGACAAGAGGGCTTCCAAAAGCCAACACT
CAGCCAGCCACTGCTCATCTGCTGACTGCCAGACTCAAAGCTACCAACAAAA

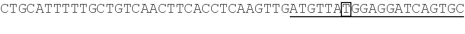

ACTAGCCTGCCACTCTGGCAAGTGTATAAGGTTGCCCAGGACTCTTCTGATATT
CCAAAATACTATTTATGTATACAATTTGAATGACACTAATATCTATATGGGAT
ACAAAAAAAAATACAAAAATGAGATTCTTCTGATGTTTTTAATATTTGTTGAT
TAAAGACACTCATCCATGGCAGCTATATAAACCTTATGCAGCAAAATATTTG
AATGGCAGCTGTTTTGGTAGCCGATGTCAAGTGCCCCCACACTCACCAACATTC
TATCTAAAGAGCGTCATGGACAAAATGGGATGTTACCCACAACACTGGTAAG
GCAGTCTACTTTCATTAACCCCCTTCCTGGCAGCAGGGCAGTGAGTGAACTAC
CAAATAAAAAACATCTACATACACCTAACCCTCCGTTATCGCGCCCTTCTGTT
ATCCGCGTTTACGCATTATCGCACACTTATGAAAATGTGCAAAATTCAGTTTTA
GCGCATCTGGAAAGTTGTTATCGC

Primer Premier 5 software was used to design primer PS8 for sex marker verification according to the sequence of scaffold325741, the primer design should meet the following conditions: the target amplicon is between 100-300 bp; the annealing temperature should be 50-60° C.; mismatches, hairpin structure and primer dimer should be avoided between the forward and reverse primers. The primers were synthesized by Sangon Biotech (Shanghai) Co., Ltd. (Table 6).

TABLE 6
Information of sequencing primers
(annealing temperature Ta: 60° C.)

| Primer ID | | Primer sequence |
|---|---|---|
| PS8 | Forward | 5′ -ATACCAGACAAGAGGGCTTC- 3′ |
| | | (SEQ ID NO: 11) |
| | Reverse | 5′ -TCCCATATAGATATTAGTGTCATTC- 3′ |
| | | (SEQ ID NO: 12) |

The KASP detection primers were designed according to the sequence information of the SNP sitePolymarker (http://www.polymarker.info) was used to design two allele-specific reverse primers (the 5′ends were respectively connected to specific sequences that can bind to FAM fluorophores and HEX fluorophores) and a universal forward primer. The primer combinations for site PtS2 (located in scaffold325741 sequence) are: PtS2FAM, PtS2HEX, PtS2C. The primer sequences are shown in Table 7.

TABLE 7
KASP primer sequence information table

| LocusID | Primer | Sequence (5′-3′) |
|---|---|---|
| PtS2 | PtS2FAM | GAAGGTGACCAAGTTCATGCTAGGCTA |
| | | GTGCACTGATCCTCCA (SEQ ID |
| | | NO: 13) |
| | PtS2HEX | GAAGGTCGGAGTCAACGGATTGGCTA |
| | | GTGCACTGATCCTCCG (SEQ ID |
| | | NO: 14) |
| | PtS2C | CTGTCAACTTCACCTCAAGTTGATGTTA |
| | | (SEQ ID NO: 15) |

Note:
FAM specific sequence: GAAGGTGACCAAGTTCATGCT;
HEX specific sequence: GAAGGTCGGAGTCAACGGATT.

1. Using Sequencing Method for Genotyping Detection

A total of 60 individuals (30 females+30 males) from a wild population of P. trituberculatus were collected, and the DNA of muscle was extracted using the tissue genomic DNA extraction kit (Bioteke, Beijing).

PS8 primers were used for PCR amplification and sequencing. At the same time, paraffin section and HE staining were performed on the sample gonad tissue to ensure the accuracy of gender identification.

The PCR reaction system is 10 μL, including: 100 ng DNA, 2×Power Taq PCR Master Mix (Bioteke, Beijing) 5 μL, 1 mol of each primer.

The PCR amplification program is: denaturation at 94° C. for 3 min; 35 cycles of denaturation at 94° C. for 1 min, 60° C. for 1 min, and 72° C. for 1 min. Finally, extend for 5 min at 72° C. and store at 4° C. The PCR products were sent to Sangon Biotech (Shanghai) Co., Ltd. for sequencing in both directions. The sequencing results showed that there was a SNP site difference between male and female. Females are homozygous and males are heterozygous. The specific genotypes are presented in Table 8, and the sequencing diagrams are presented in FIG. 5.

TABLE 8
Genotype sequencing results of male and female
individuals of P. trituberculatus

| Primer name | Genotype | Male | Female | Specificity (%) |
|---|---|---|---|---|
| PS8 | T/C | 30 | 0 | 100.0 |
| | T/T | 0 | 30 | |

2) Using KASP Method for Genotyping Detection

In addition, 92 individuals (46 females+46 males) from the wild population of P. trituberculatus were collected, and the genomic DNA of the muscle tissue of P. trituberculatus was extracted according to the steps of the EZNA® tissue DNA kit instructions.

The PtS2 site was amplified and sequenced by PCR using the KASP primer combination. At the same time, paraffin section and HE staining were performed on the sample gonad tissue to ensure the accuracy of gender identification.

KASP reaction was carried out on Hydrocycler-16 (LGC Genomics, UK), the reaction system was including 0.5 μl×KASP 1536 Master Mix, 0.014W Primer mix (Primer mix includes 46 μl ddH₂O, 30 μl common primer (100 μM), 12 μ 1 each of allele-specific primers (100 μM),), 10 ng DNA. The Touch down PCR program is as follows: 94° C. for 10 min; 10 touch-down cycles at 94° C. for 20 s, 61-55 C for 10 s (−0.6° C. per cycle); 26 cycles at 94° C. for 20 s, 55° C. for 60s. Pherastar scanner (LGC Genomics, UK) was used for fluorescence detection of the reaction, and Kraken software (LGC Genomics, UK) was used for data analysis. If no genotyping is observed after the initial amplification, another 5-10 cycles could be added and analyzed again.

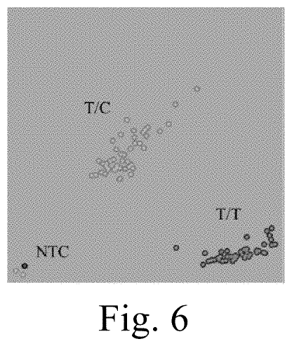

The results of genotyping 92 wild P. trituberculatus using KASP method are shown in FIG. 6. The typing results of PtS2 are homozygous T/T (blue dots) and heterozygous T/C (green dots). The genotyping results of PtS2 are consistent with the phenotypic sex of eachindividual, and conform to the law of homozygous female and heterozygous male. The PtS2 locus was successfully amplified in 90 individuals (2 female individuals failed to be amplified), and the accuracy rate of successfully amplified individuals was 100%.

The above results indicate that the SNP sites selected by the present invention can be used for sex identification of P. trituberculatus.

What is claimed is:

1. A Single nucleotide polymorphism (SNP) markers for genetic sex identification of Portunus trituberculatus, comprising a first SNP site of a fragment of SEQ ID NO:1; or by substituting, deleting, or adding one or more nucleotides from the SEQ ID NO:1; wherein the SNP site is a complementary fragment of the fragment SEQ ID NO:1, position 1452, which is 1452A>G; Or
a second SNP sites comprising six sex-linked SNP sites distributed on the fragment of SEQ ID NO: 7, comprising 153C>T, 185G>A, 214T>C, 236T>(A, C), 251T>C, 261A>T; Or
a third SNP site comprising a sex-linked SNP site distributed at position 878 of SEQ ID NO: 10, and its base is T>C.

2. A method for detecting the genetic male and female sex of P. trituberculatus comprising: distinguishing the genetic male and female sex of P. trituberculatus by detecting the SNP site of claim 1.

3. The method according to claim 2, wherein the method is detected by PCR amplification and sequencing methods, and the sequencing result is compared with the molecularly labeled DNA sequence, if the sequencing peak pattern of the tested individual is consistent with the molecularly labeled DNA sequence, and the SNP position is a single peak, the tested individual is female, and if the sequencing peak diagram exhibits double peaks at the SNP position, it is male.

4. The method according to claim 2, wherein the method is to detect by the KASP method, and determine the genotype of the sex-linked locusof P. trituberculatus based on the fluorescent signal to identify the genetic sex of the sample.

5. The method according to claim 2, wherein the method is to detect by a high-resolution melting curve detection method.

6. The method according to claim 3, wherein the PCR amplification and sequencing methods are used for detection, wherein the primer pair used for PCR amplification and sequencing for detecting the first SNP site, the sequence of the upstream and downstream primers, these are SEQ ID NO: 2 and SEQ ID NO: 3.

7. The method according to claim 3, wherein the PCR amplification and sequencing methods are used for detection, wherein the PCR amplification and sequencing primer pair used to detect the third SNP site, the upstream and downstream primers, the sequences are SEQ ID NO: 11 and SEQ ID NO: 12.

8. The method according to claim 4, wherein the first SNP site is detected by the KASP method, wherein the sequences of the primers used are SEQ ID NO: 4, SEQ ID NO: 5, SEQ ID NO: 6.

9. The method according to claim 4, wherein the third SNP site is detected by the KASP method, wherein the sequences of the primers used are SEQ ID NO: 13, SEQ ID NO: 14, SEQ ID NO: 15.

10. The method according to claim 5, wherein the resolution melting curve detection method, wherein the primer pair used to detect the PCR amplification and sequencing of the second SNPmarker are SEQ ID NO: 8 and SEQ ID NO: 9.

11. The method according to claim 10, wherein the peak value of the male melting curve detected by the method is at 80.52° C., and the peak value of the female melting curve is located at 81.91° C.

12. A PCR detection product for genetic sex identification of P. trituberculatus, wherein the PCR detection product is used for detecting the SNP site of claim 1.

13. The PCR detection product of claim 12, wherein the PCR detection product is a PCR amplification and sequencing detection product.

14. The PCR detection product of claim 13, wherein the detection product contains a primer pair for detecting PCR amplification and sequencing of the first SNP site of claim 1, on which the sequences of the downstream primers are SEQ ID NO: 2 and SEQ ID NO: 3.

15. The PCR detection product of claim 13, wherein the detection product contains a primer pair for PCR amplification and sequencing for detecting the third SNP site of claim 1, on which the sequences of the downstream primers are SEQ ID NO:11 and SEQ ID NO:12.

16. The PCR detection product of claim 12, wherein the PCR detection product is a KASP method detection product.

17. The PCR detection product of claim 16, wherein the PCR detection product contains a primer set for detecting the first SNP site, the sequence of which are SEQ ID NO: 4, SEQ ID NO :5, SEQ ID NO:6.

18. The PCR detection product of claim 16, wherein the PCR detection product contains a primer set for detecting the third SNP site, the sequence of which are SEQ ID NO: 13, SEQ ID NO : 14. SEQ ID NO: 15.

19. The PCR detection product of claim 12, wherein the PCR detection product is a high-resolution melting curve detection product.

20. The PCR detection product of claim 19, wherein the PCR detection product comprises a primer for detecting the second SNP sites, the sequence of which are SEQ ID NO: 8 and SEQ ID NO: 9.
